(19) United States
(12) Patent Application Publication
(10) Pub. No.: US 2023/0181237 A1
(43) Pub. Date: Jun. 15, 2023

(54) SURGICAL DEVICE CONNECTION HUB

(71) Applicant: GYRUS MEDICAL LIMITED, Cardiff (GB)

(72) Inventor: Wilhelmus GOMMEREN, Cardiff (GB)

(73) Assignee: GYRUS MEDICAL LIMITED, Cardiff (GB)

(21) Appl. No.: 18/073,704

(22) Filed: Dec. 2, 2022

Related U.S. Application Data
(60) Provisional application No. 63/288,917, filed on Dec. 13, 2021.

(51) Int. Cl. A61B 18/14; A61B 17/28

(52) U.S. Cl. CPC A61B 18/1445; A61B 17/28; A61B 2018/0063

(57) ABSTRACT

The present invention relates to an electrosurgical device wherein a surgical instrument is connectable to a handpiece via a connection hub, the connection hub facilitating an electrical and mechanical connection therebetween. The connection hub is configured to implement a cam-follower mechanism for securing an electrical connection between the surgical instrument and a handpiece configured to receive the surgical instrument. As a result of such a configuration, the electrical connection can be established in a more efficient manner compared to prior art implementations.

### FIELD OF DISCLOSURE

The present invention relates to a connection hub for a surgical instrument. In particular, it relates to a connection hub implementing a cam-follower mechanism for establishing an electrical connection between a surgical instrument and a handpiece configured to receive the surgical instrument.

### BACKGROUND

Electrosurgical instruments provide advantages over traditional surgical instruments in that they can be used for coagulation and tissue sealing purposes. One such prior art arrangement is known from U.S. Pat. No. 5,904,681 which describes a surgical instrument including a mechanical cutting portion, such as a rotary blade or burr, and a radio frequency (RF) cutting and/or cauterizing portion comprising an electronic surgical device which operates in bipolar mode.

Another prior art arrangement is known from U.S. Pat. No. 9,017,851 which describes an apparatus for powering a medical device, including a protective layer covering a battery pack and a connection feature. The connection feature provides a fluid tight seal, for example by having an electrode which may pierce the protective layer to establish electrical communication with the medical device from within the protective layer, thereby allowing a non-sterile battery pack to deliver power to a sterile medical device.

Another prior art arrangement is known from EP0746251B1 which describes an integrated catheter assembly including a catheter having an irrigation fluid lumen, a distal tip portion having electrodes, and a needle extending through the catheter lumen and a lumen in the distal tip portion. The catheter is carried by a needle hub structure which provides a sealing

30 entrance for a needle that can be displaced between extended and retracted positions while being electrically isolated.

Another prior art arrangement is known from US2009/221955A1 which describes an ablative apparatus having a distal end at which an ablation probe driven by a transducer 35 may be vibrated to ablate tissues.

### SUMMARY OF DISCLOSURE

Embodiments of the present invention relate to providing an efficient means for establishing an electrical connection between a surgical instrument and a handpiece configured to receive the surgical instrument. In particular, the present invention relates to a connection hub for a surgical instrument, where the connection hub is configured to establish an electrical connection between the surgical instrument and the handpiece.

A surgical device, for example, an electrosurgical device comprises a surgical instrument connected to a handpiece via a connection hub disposed at a proximal end of the surgical instrument. Prior art implementations of a connection hub for a surgical instrument have the drawback of requiring a secondary action for establishing an electrical connection between the surgical instrument and the handpiece, after a mechanical connection between the instrument and handpiece has been established.

The inventors have recognised the need to provide a simple and efficient connection hub configuration which addresses the above problem and enables not only a mechanical connection but also an electrical connection to be established between a surgical device and a handpiece.

The connection hub of the present invention is configured to implement a cam-follower mechanism for securing an electrical connection between the surgical instrument and a handpiece configured to receive the surgical instrument. As a result of such a configuration, the electrical connection can be established in a more efficient manner compared to prior art implementations. For example, as will also be described in detail in the description below, a locking mechanism for establishing a mechanical connection between the surgical instrument and the handpiece can also advantageously be used to actuate the rotation of the cam in the connection hub in order to establish an electrical connection between the surgical instrument and the handpiece—for example, a linear actuation could be transposed into the said rotation of the cam.

According to an aspect of the invention, a connection hub for connecting a surgical instrument to a handpiece is provided, wherein the connection hub comprises: a first member; a second member, wherein the first member and the second member are disposed coaxially relative to a longitudinal axis of the surgical instrument, the second member being disposed within the first member, wherein the first member comprises at least one flexible connector disposed on an outer surface of the first member, wherein the second member comprises a cam feature and wherein one of the first member or the second member is configured to be rotatable relative to the other of the first member or the second member such that, when one of the first member or the second member is rotated in a first direction relative to the other of the first member or the second member, the cam feature is configured to exert a force on the at least one flexible connector.

According to a further aspect of this invention a surgical instrument is provided, wherein the surgical instrument is configured to be releasably connectable to a handpiece and wherein the surgical instrument comprises: an elongate shaft; an end effector at a distal end of the elongate shaft, and a connection hub, as described above, at a proximal end of the elongate shaft.

According to an aspect of this invention a surgical device is provided comprising a surgical instrument as specified above and a handpiece configured to be releasably connected to the surgical instrument.

According to another aspect of this invention, a surgical device is provided comprising: a handpiece; a surgical instrument configured to be releasably connected to the handpiece, the surgical instrument comprising an elongate shaft having a connection hub at a proximal end and an end effector at a distal end, wherein the handpiece is configured to receive the connection hub and wherein the connection hub is configured to implement a cam-follower mechanism such that rotating the cam portion exerts a force on a flexible connector disposed on the connection hub to establish a contact between the flexible connector on the connection hub and a corresponding connector disposed on the handpiece. In some embodiments, the hub connection may incorporate a mechanical connection for a drive system.

According to another aspect of this invention, an electrosurgical system is provided wherein the electrosurgical system comprises of but not limited to: a radiofrequency electrosurgical generator; and a surgical device as specified above, wherein the surgical instrument is configured to be an electrosurgical instrument.

### BRIEF DESCRIPTION OF THE DRAWINGS

The teachings of this disclosure will be discussed, by way of non-limiting examples, with reference to the accompanying drawings, in which:

FIG. 1a shows an example implementation of the connection hub according to an embodiment of this disclosure.

FIG. 1b shows an example implementation of the connection hub according to an embodiment of this disclosure, where the inner member of the connection hub is rotated relative to the outer member.

FIG. 2 shows an example implementation of a handpiece configured to receive a connection hub of a surgical instrument.

FIG. 3 shows an example implementation of an electrosurgical system comprising an electrosurgical device according to an embodiment of this disclosure.

### DETAILED DESCRIPTION

A surgical instrument, in particular, an electrosurgical instrument can be used to perform different operations, including mechanical cutting of tissue, and RF electrosurgical ablation, sealing and/or coagulation of tissue, with mechanical cutting and RF electrosurgical ablation, sealing or coagulation of tissue capable of taking place simultaneously. An electrosurgical device comprises a surgical instrument releasably connected to a handpiece, where the handpiece can be configured to power the surgical instrument electrically.

The present invention relates to providing an efficient means for establishing an electrical connection between a surgical instrument and a handpiece configured to receive the surgical instrument. In particular, the present invention relates to a connection hub for a surgical instrument, where the connection hub is configured to establish an electrical and mechanical connection between the surgical instrument and the handpiece. The connection hub of the present invention is configured to implement a cam-follower mechanism for securing an electrical connection between the surgical instrument and a handpiece configured to receive the surgical instrument.

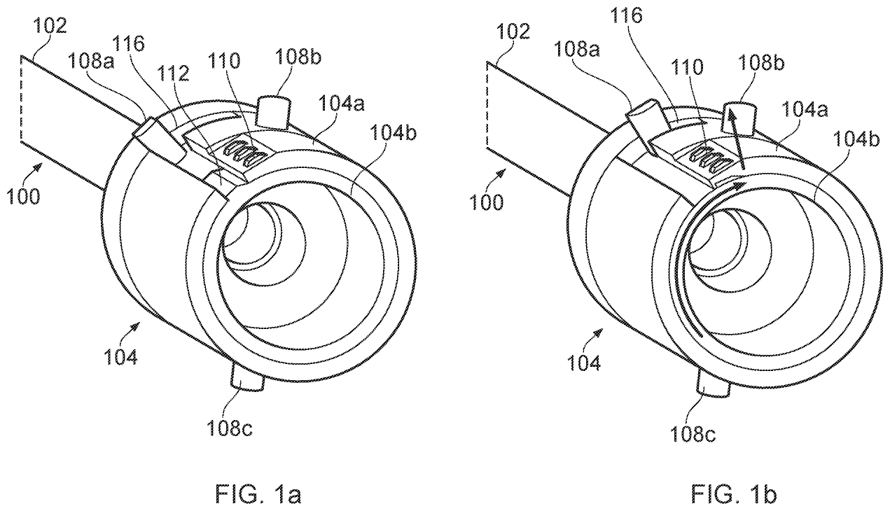

FIG. 1a shows an example implementation of the connection hub 104 according to an embodiment of this disclosure. The connection hub 104 can be disposed at or near a proximal end of an elongate shaft 102 of the electrosurgical instrument 100. The electrosurgical instrument 100 comprises an end-effector at its distal end (not shown).

In the example shown in FIG. 1a, the connection hub 104 comprises an outer member 104a and an inner member 104b which are disposed coaxially with respect to a longitudinal axis of the elongate shaft 102. The inner member 104b comprises a cam feature 112. The outer member comprises at least one non-rigid or flexible electrical connector 110. The outer member 104a is configured to be rotatable relative to the inner member 104b or vice versa, such that, when one of the outer member 104a or the inner member 104b is rotated in a first direction relative to the other member, the cam feature is configured to exert a force on the at least one flexible electrical connector 110 to push the flexible electrical connector 110 in an outward direction.

FIG. 1b shows the inner member 104b rotated relative to the outer member 104a. As seen in FIG. 1b, the cam feature 112 on the outer surface of the member 104b is configured to translate a rotational motion of the inner member 104b to a linear motion, exerting a force on flexible electrical connector 110 so as to push the connector in an outward direction.

In some embodiments, the outer member 104a may be configured to be a static member, fixed in relation to the elongate shaft 102 of the electrosurgical instrument 100, and the inner member 104b may be configured to be rotatable relative to the outer member 104a. That is, the static member 104a may be configured such that it does not rotate relative to the elongate shaft 102.

The connection hub 104 may comprise a locking mechanism configured to lock the inner member 104b to the outer member 104a. For example, as shown in FIG. 1a, the inner member 104b may comprise a locking tab 108a configured to project from an outer circumferential surface of the inner member 104b. The outer member 104a may comprise a slot 116 configured to engage with the locking tab 108a when the inner member 104b is rotated in a first direction relative to the elongate shaft 102. The slot 116 may be configured to engage with the locking tab 108a so as to restrict the rotation of the inner member 104b along the first direction relative to the elongate shaft 102.

As shown in the example implementation in FIG. 1a, the outer member 104a of the connection hub 104 may comprise at least one tab 108b which may protrude from an outer circumferential surface of the outer member 104a. A handpiece configured to be releasably connected to the surgical instrument 100 via the connection hub 104 may comprise a corresponding slot for receiving the at least one tab 108b on the outer member 104a of the connection hub. The implementation of this lock and key arrangement will be described in detail later in this description when describing the configuration of the handpiece 120. In some embodiments, the outer member 104a may comprise two or more tabs 108b, 108c. The two tabs 108b, 108c may have different dimensions. For example, the first tab 108b may have a greater width than the second tab 108c.

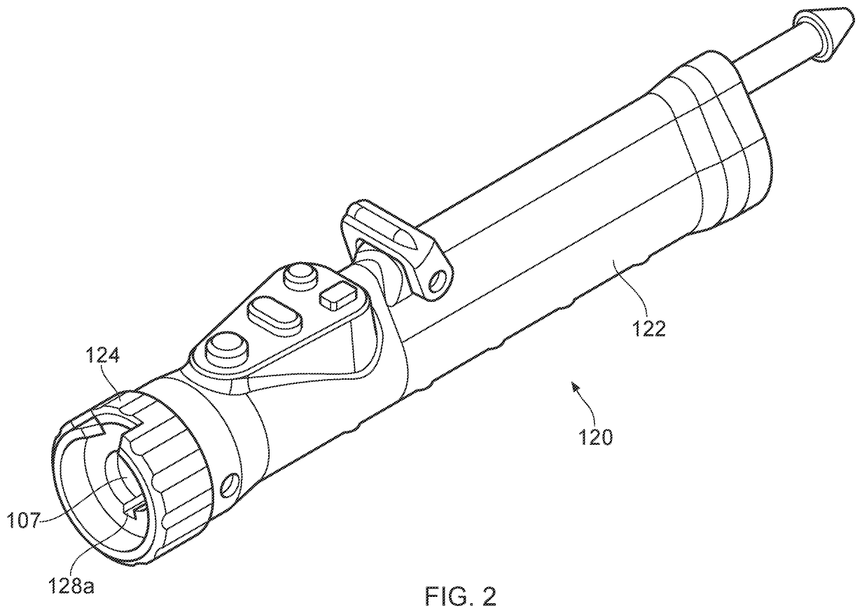

The handpiece 120, as shown in FIG. 2, comprises a main body 122 which may have activation buttons thereon to permit an operator to activate the mechanical cutting or electrosurgical operations using the electrosurgical instrument 100. The handpiece 120 comprises an axial chamber 107 defined within the main body 122, where the axial chamber 107 is configured to receive the electrosurgical instrument 100. The handpiece 120 further comprises a rotatable feature 124 which can be, for example, a collet which is substantially annular and extends from the handpiece in a longitudinally axial direction therefrom. The rotatable feature 124 is rotatable relative to the handpiece 100.

The handpiece 120 may be configured to implement a locking mechanism to engage or release the electrosurgical instrument 100 based on the rotation of the collet 124 relative to the handpiece 120. In some embodiments, the handpiece 120 may comprise at least one slot 128a configured to receive the at least one locking tab 108b (see FIG. 1a) on the connection hub 104 of the electrosurgical instrument 100. When the collet 124 is rotated in a first direction relative to the handpiece 120, the electrosurgical instrument 100 may be inserted into the handpiece 120 by inserting the at least one locking tab 108b into the corresponding slot 128a in the handpiece. In this way, a locking mechanism is implemented which enables the electrosurgical instrument 100 to be secured to the handpiece 120. When the collet 124 is rotated in a second direction relative to the handpiece 120, the electrosurgical instrument is disengaged, for example, by disengaging the locking tab 108b from the corresponding slot 128a, in order to enable the removal of the electrosurgical instrument 100 from the handpiece 120. In some embodiments, the electrosurgical instrument could be rotated instead of the collet 124 to fix on a feature on the handpiece.

In some embodiments, upon insertion of the electrosurgical instrument 100 into the handpiece 120, the above-described rotation of the outer member 104a or the inner member 104b of the connection hub 104 may be actuated by the rotation of the collet 124 or rotation of the instrument. That is, the collet 124 may be rotated in a first direction relative to the handpiece to secure the electrosurgical instrument 100 to the handpiece 120 as well as to rotate the outer member 104a or the inner member 104b of the connection hub 104 in order to enable the flexible electrical connector 110 to contact corresponding connectors (not shown) disposed on the handpiece 120. In other embodiments, the electrosurgical instrument 100 may comprise an actuator configured to actuate the above-described rotation of the outer member 104a or the inner member 104b of the connection hub 104, upon insertion of the electrosurgical instrument 100 in the handpiece 120, in order to enable the flexible connectors 110 on the connection hub 104 to contact corresponding connectors disposed on the handpiece 120.

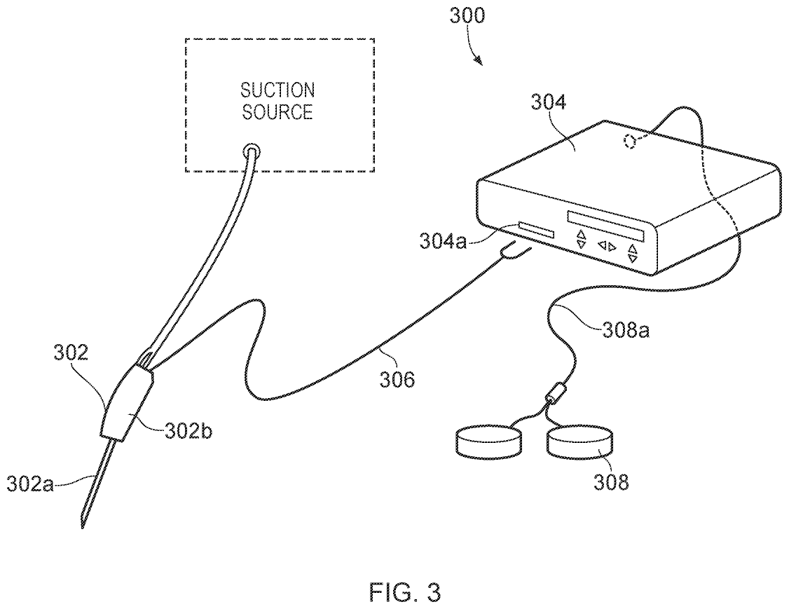

FIG. 3 shows an example of an electrosurgical system 300 comprising an electrosurgical device 302 according to an embodiment of the present disclosure. The electrosurgical system 300 comprises an electrosurgical generator 304 having one or more sockets 304a providing a radio frequency (RF) output, via a connection cord 306, for the electrosurgical device 302. The electrosurgical device 302 comprises an electrosurgical instrument 302a releasably connected to a handpiece 302b via a connection hub (not shown). The connection hub is configured according to the embodiments of this disclosure as specified above.

Activation of the generator 304 may be performed from the device 302 via a hand-switch (not shown) on the device 302, or by means of a footswitch unit 308 connected separately to the generator 304 by a footswitch connection cord 308a. In some embodiments, the footswitch unit 308 could be connected to the generator by wireless means. In another embodiment, the electrosurgical device 302 may include the means to generate electrosurgical signals, for example, using an electrosurgical generator incorporated into the device.

While the present disclosure refers to electrosurgical instruments, devices and systems, it

will be appreciated that the disclosure is also applicable to any surgical instrument, device

or system having an electrically powered end effector connectable to a handpiece. In other embodiments, the arrangement can also be applicable to end effectors which are powered by other modes, such as pneumatically or hydraulically. Although the arrangement has been described with reference to a substantially circular cross-section, a similar arrangement for a different shape could be envisaged. The electrosurgical instrument may be a disposable instrument while the handpiece may be re-usable.

It will be appreciated that various modifications, whether by way of addition, deletion and/or substitution, may be made to all of the above described embodiments to provide further embodiments, any and/or all of which are intended to be encompassed by the appended claims.

1. A connection hub for connecting a surgical instrument to a handpiece, the connection hub comprising:
a first member;
a second member,
wherein the first member and the second member are disposed coaxially relative to a longitudinal axis of the surgical instrument, the second member being disposed within the first member,
wherein the first member comprises at least one flexible connector disposed on an outer surface of the first member,
wherein the second member comprises a cam feature and
wherein one of the first member or the second member is configured to be rotatable relative to the other of the first member or the second member such that, when one of the first member or the second member is rotated in a first direction relative to the other of the first member or the second member, the cam feature is configured to exert a force on the at least one flexible connector.

2. A connection hub according to claim 1, wherein the first member is configured to be a static member and wherein the second member is configured to be rotatable relative to the first member.

3. A connection hub according to claim 1, wherein the at least one flexible connector comprises an electrical connector.

4. A connection hub according to claim 1, wherein the connection hub comprises a first locking arrangement such that when the one of the first member or the second member is rotated in a first direction relative to the other of the first member or the second member, the first locking arrangement is configured to lock the first member to the second member.

5. A connection hub according to claim 4 wherein the first locking arrangement comprises:
a first locking tab on the outer surface of the second member; and
a slot in the first member configured to receive the first locking tab of the second member.

6. A connection hub according to claim 1, wherein the connection hub comprises a substantially circular cross-section.

7. A surgical device, configured to be releasably connectable to a handpiece via a connection hub, the surgical device comprising:
an elongate shaft;
an end effector at a distal end of the elongate shaft, and
a connection hub at a proximal end of the elongate shaft for releasably connecting the surgical device to a handpiece, the connection hub comprising:
a first member;
a second member,
wherein the first member and the second member are disposed coaxially relative to a longitudinal axis of the surgical instrument, the second member being disposed within the first member,
wherein the first member comprises at least one flexible connector disposed on an outer surface of the first member,
wherein the second member comprises a cam feature and
wherein one of the first member or the second member is configured to be rotatable relative to the other of the first member or the second member such that, when one of the first member or the second member is rotated in a first direction relative to the other of the first member or the second member, the cam feature is configured to exert a force on the at least one flexible connector.

8. A surgical device according to claim 7, wherein the surgical device is an electrosurgical instrument.

9. A surgical device according to claim 7, and further comprising a handpiece comprising a main body configured to receive the connection hub, wherein the handpiece is configured to be releasably connected to the connection hub.

10. A surgical device according to claim 9,
wherein the handpiece comprises at least one connector disposed on a surface of the handpiece, and
wherein the surgical device is configured such that when the one of the first member or the second member of the connection hub is rotated relative to the other of the first member or the second member of the connection hub, the at least one flexible connector on the connection hub is brought into contact with at least one connector disposed on the surface of the handpiece.

11. A surgical device according to claim 9, wherein the first member of the connection hub further comprises at least one tab on the outer surface of the first member and wherein the handpiece comprises at least one slot configured to receive the at least one tab on the outer surface of the first member.

12. A surgical device according to claim 9,
wherein the handpiece further comprises a rotatable feature at a proximal end, the rotatable feature being rotatable relative to the handpiece and being configured to enable the surgical instrument to interlock with the hand piece, and
wherein the handpiece is configured to engage with the surgical instrument when the rotatable feature is rotated in a first direction relative to the handpiece and release the surgical instrument when the rotatable feature is rotated in a second direction, opposite to the first direction, relative to the handpiece.

13. A surgical device according to claim 12,
wherein the handpiece is further configured to control the rotation of one of the first member or the second member of the connection hub relative to the other of the first member or the second member of the connection hub, based on the rotation of the rotatable feature or the rotation of the surgical instrument relative to the handpiece.

14. A surgical device according to claim 9,
wherein the surgical instrument comprises an actuator for controlling the rotation of one of the first member or the second member of the connection hub relative to the other of the first member or the second member of the connection hub, and

15. A surgical device according to claim 8, and further comprising a radiofrequency electrosurgical generator.

16. A surgical device comprising:
a handpiece;
a surgical instrument configured to be releasably connected to the handpiece, the surgical instrument comprising an elongate shaft having a connection hub at a proximal end and an end effector at a distal end,
wherein the handpiece is configured to receive the connection hub and
wherein the connection hub is configured to implement a cam-follower mechanism such that rotating the cam portion exerts a force on a flexible connector disposed on the connection hub to establish a contact between the flexible connector on the connection hub and a corresponding connector disposed on the handpiece.
